(19) United States
(12) Patent Application Publication
(10) Pub. No.: US 2007/0224657 A1
(43) Pub. Date: Sep. 27, 2007

(54) Distribution of PON1 as a marker of lipid related disorders

(76) Inventors: Michael Aviram, Kiryat-Haim (IL); Mira Rosenblat, Haifa (IL)

Correspondence Address: Martin D. Moynihan;PRTSI, Inc., P.O. Box 16446, Arlington, VA, 22215, US

(21) Appl. No.: 11/580,954

(22) Filed: Oct. 16, 2006

Related U.S. Application Data
(60) Provisional application No. 60/786,004, filed on Mar. 27, 2006.

(51) Int. Cl. C12Q 1/26

(52) U.S. Cl. 435/25

(57) ABSTRACT

A method of diagnosing a subject with a lipid related disorder is disclosed, the method comprising determining an amount or activity of PON1 or apo-A1 in an LPDS fraction of a serum of the subject, wherein an amount or activity of PON1 above a predetermined threshold is indicative of the lipid related disorder. Kits for diagnosing the lipid related disorders are also disclosed.

### RELATED APPLICATION

This Application claims the benefit of U.S. Provisional Patent Application No. 60/786,004 filed Mar. 27, 2006, the contents of which are included herein.

### FIELD AND BACKGROUND OF THE INVENTION

The present invention relates to a method of diagnosing lipid related disorders based on the distribution of PON1 from HDL to lipoprotein deficient serum.

Atherosclerosis is a disorder characterized by cellular changes in the arterial intima and the formation of arterial plaques containing intracellular and extracellular deposits of lipids. The thickening of artery walls and the narrowing of the arterial lumen underlies the pathologic condition in most cases of coronary artery disease, aortic aneurysm, peripheral vascular disease, and stroke. Another major disease associated with atherosclerosis is diabetes.

Paraoxonase 1 (PON1) is an HDL-associated esterase/lactonase and its activity is inversely related to the risk of cardiovascular diseases. Recently, PON1 was shown to be associated also with triglyceride-rich lipoproteins (chylomicrons and VLDL), but not with LDL. The crystal structure elucidation of a variant of PON1 obtained by directed evolution showed that PON1 consists of a six-bladed β propeller with a unique active site. The role of PON1 in atherosclerosis development was demonstrated in studies, which used mice lacking PON1, or overexpressing PON1 [11, 12]. PON1 antiatherogenic properties include protection of low density lipoprotein (LDL), high density lipoprotein (HDL) and macrophages against oxidative stress, attenuation of oxidized-LDL uptake by macrophages, inhibition of macrophage cholesterol biosynthesis, and stimulation of HDL-mediated cholesterol efflux from macrophages. Among HDL subfractions, HDL₃, which is important in reverse cholesterol transport, carries the highest PON1 activity.

Most of serum PON1 is localized on the surface of HDL, and HDL major apolipoprotein, A-I (apoA-I) was shown to stabilize PON1 activity [1, 2]. Under pathological conditions, such as in patients with low plasma apoA-I levels, PON1 distributed from small-size HDL to the LPDS (lipoprotein deficient serum) [20]. In human apoA-I deficiency, 38% of PON1 protein was found in the lipoprotein-free fraction, whereas in healthy subjects only 5% of total serum PON1 protein was in LPDS [21]. In humans and rabbits most of the PON1 arylesterase activity was shown to be HDL-associated, whereas in mice 30% of this activity was found in LPDS [22]. In the absence of apoA-I in mice, total PON1 arylesterase activity was reduced and over 60% was found in LPDS [22]. PON1 arylesterase activity and distribution were restored in apoA-I deficient mice following injection of adenoviruses encoding human apoA-I [22].

US Patent Application No. 20030027759 teaches a method of decreasing an atheroma by treating with PON1. US Patent Application No. 20030027759 however, does not teach diagnosing lipid related disorders by analyzing levels of PON1.

Due to their high incidence and high variability in treatment response, there still remains a widely recognized need for, and it would be highly advantageous to have more effective indicators for diagnosing atherosclerosis and related diseases, as well as more effective indicators for monitoring the course of such diseases.

### SUMMARY OF THE INVENTION

According to one aspect of the present invention there is provided a method of diagnosing a subject with a lipid related disorder, the method comprising determining an amount or activity of PON1 in an LPDS fraction of a serum of the subject, wherein an amount or activity of PON1 above a predetermined threshold is indicative of the lipid related disorder.

According to another aspect of the present invention there is provided a method of diagnosing a subject with a lipid related disorder, the method comprising determining an amount of apo-A1 in an LPDS fraction of a serum of the subject, wherein an amount of apo-A1 above a predetermined threshold is indicative of the lipid related disorder.

According to yet another aspect of the present invention there is provided a method of diagnosing a subject with a lipid related disorder, the method comprising determining a ratio of an amount or activity of PON1:HDL to PON1:LPDS in a serum of the subject, wherein a ratio below a predetermined threshold is indicative of the lipid related disorder.

According to still another aspect of the present invention there is provided a kit for diagnosing or determining a predisposition to a lipid related disorder, the kit comprising at least one agent capable of determining an amount or activity of PON1 in a LPDS fraction of a serum sample and instructions for carrying out the diagnosing in the LPDS fraction.

According to further features in preferred embodiments of the invention described below, the method further comprises determining an amount or activity of PON1 in an HDL fraction of the serum.

According to still further features in the described preferred embodiments, the activity of PON1 is a paraoxonase activity.

According to further features in preferred embodiments of the invention described below, the method further comprises determining an amount of apo-A1 in the LPDS fraction of the serum of the subject.

According to further features in preferred embodiments of the invention described below, the method further comprises determining an amount of apo-A1 in an HDL fraction of said serum

According to still further features in the described preferred embodiments, the lipid related disorder is selected from the group consisting of diabetes mellitus, atherosclerosis, coronary heart disease, myocardial infarction, peripheral vascular diseases, venous thromboembolism and pulmonary embolism.

According to still further features in the described preferred embodiments, the lipid related disorder is diabetes mellitus.

According to further features in preferred embodiments of the invention described below, the method further comprises determining an amount or activity of PON1 in the LPDS fraction of the serum of the subject.

According to further features in preferred embodiments of the invention described below, the method further comprises determining an amount or activity of PON1 in an HDL fraction of the serum.

According to further features in preferred embodiments of the invention described below, the method further comprises determining a ratio of an amount of apo-A1:HDL to apo-A1:LPDS in the serum of the subject.

The present invention successfully addresses the shortcomings of the presently known configurations by providing a novel method of diagnosing lipid related disorders.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the patent specification, including definitions, will control. In addition, the materials, methods, and examples are illustrative only and not intended to be limiting.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIGS. 1A-C are graphs indicating the levels of paraoxonase activities in FPLC fractionated serum from healthy subjects. Serum samples (100 μl diluted X2) from 3 healthy subjects were fractionated by FPLC and sixty fractions of 0.5 ml each were collected and the protein absorbance was monitored at 260 nm (FIG. 1A), Arylesterase (FIG. 1B) and paraoxonase (FIG. 1C) activities were measured in all fractions. Results are given as mean±SD (n=3).

FIGS. 2A-C are bar graphs indicating the levels of paraoxonase activities in the HDL and in the LPDS fractions separated from the serum of healthy subjects by density gradient ultracentrifugation. The serum samples (3.5 ml) of 3 healthy subjects were fractionated and paraoxonase (FIG. 2A), arylesterase (FIG. 2B), and lactonase (FIG. 2C) activities were measured in the HDL and LPDS fractions. Results represent the mean±SD (n=3), and are given as units in fraction which originated from 1 ml of serum. *p<0.01 vs. HDL.

FIGS. 3A-D are bar graphs comparing HDL and LPDS PON1 paraoxonase activity and protein in fractions isolated from the serum of healthy subjects and diabetic patients. The HDL and LPDS fractions were isolated from the serum of 3 healthy subjects (Controls) or from 3 diabetic patients by FPLC or by density gradient ultracentrifugation (UC). Paraoxonase activity was determined in the FPLC HDL and LPDS fractions (FIG. 3A), as well as in the UC fractions (FIG. 3B). PON1 protein was analyzed by Western blot analysis, and the densitometric analysis of the protein bands, as well as the bands pictures are given for the FPLC (FIG. 3C) and UC (FIG. 3D) fractions. HDL-C: Control HDL, HDL-D: diabetic HDL, LPDS-C: Control LPDS, LPDS-D; Diabetic LPDS. Results are presented as mean±SD. #p<0.01 Diabetic HDL vs. Control HDL. *p<0.01 Diabetic LPDS vs. control LPDS.

FIGS. 4A-B are bar graphs illustrating the effect of HDL or LPDS enrichment with PON1 on their susceptibility to AAPH-induced lipids peroxidation. FIG. 4A: HDL and LPDS (5 paraoxonase units/ml) were incubated with evolved PON1 (50 paraoxonase U/ml), as well as with the PON1 vehicle solution for 2 hours at 37° C. The HDL and LPDS samples were then incubated with or without 100 mM AAPH for 2 hours at 37° C. The extent of AAPH-induced lipid peroxidation was measured by the lipid peroxides assay and calculated as described under the Methods section. FIG. 4B: HDL and LPDS (5 paraoxonase units/ml) were incubated with evolved PON1 (50 paraoxonase units/ml) for 2 hours at 37° C., and paraoxonase activity was determined at the end of the incubation period. The calculated values are the sum of the values obtained for HDL or LPDS alone+added PON1 activity. Results are given as mean±S.D of three different experiments. *p<0.01 vs. HDL.

FIG. 5 is a bar graph illustrating the difference in the ability of HDL and LPDS (derived from healthy subjects) to induce macrophage cholesterol efflux: effect of PON1 enrichment. HDL and LPDS were isolated from 3 healthy subjects by ultracentrifugation. J774 A.1 macrophages were and incubated at 37° C. for 1 hour with [³H]-cholesterol (2 μCi/ml) following by cell wash and a further incubation for 3 hours at 37° C. with or without non-treated HDL fractions (100 μg of protein/ml) or with non-treated LPDS fractions (at similar paraoxonase activity as in HDL). The cells were also incubated with HDL or LPDS fractions that were preincubated for 1 hour at 37° C. with the PON1 inhibitor 2-hydroxyquinoline (200 μM), or with HDL and LPDS that were enriched with evolved PON1 (50 paraoxonase U/ml). HDL or LPDS-mediated cholesterol efflux was then determined. Results are given as mean±S.D of three different experiments *p<0.01 vs. HDL, #p<0.01 vs. LPDS.

### DESCRIPTION OF THE PREFERRED EMBODIMENTS

The present invention is of a method of diagnosing lipid-related disorders such as Diabetes.

The principles and operation of the method according to the present invention may be better understood with reference to the drawings and accompanying descriptions.

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

Paraoxonase 1 (PON1) is an HDL-associated esterase/lactonase. It comprises anti-atherogenic and detoxification properties, hydrolyzing a wide range of substrates, such as esters, organophosphates (e.g., paraoxon) and lactones. Recently, PON1 was shown to associate with lipoproteins other than HDL such as triglyceride-rich lipoproteins (chylomicrons and VLDL). An absence of arylesterase activity in lipoprotein deficient serum (LPDS), led to the popular belief that PON1 would always be found associated with lipoproteins and that free, unbound PON-1 did not exist [22].

Apolipoprotein AI (apo AI) is the major protein component of the serum high density lipoprotein (HDL) particles and is responsible for binding PON1 to the HDL.

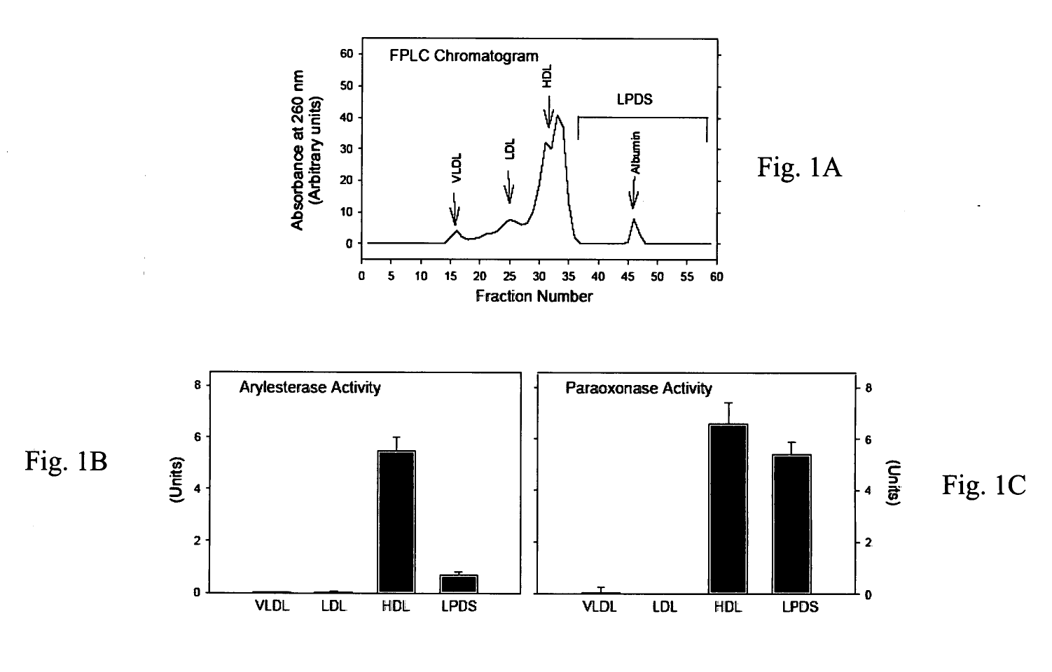

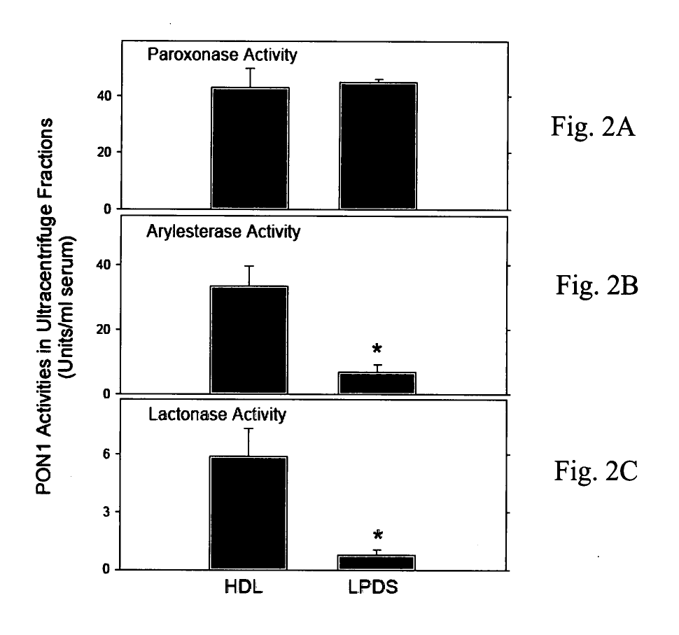

The present invention is based on the unexpected finding that free, unassociated PON1 does indeed exist. The present inventors have shown that although PON1 loses its arylesterase and lactonase activities in LPDS, it maintains its paraoxonase activity. This is in sharp contrast to lipoprotein associated PON1, which comprises all three activities (FIGS. 1A-C and FIGS. 2A-C).

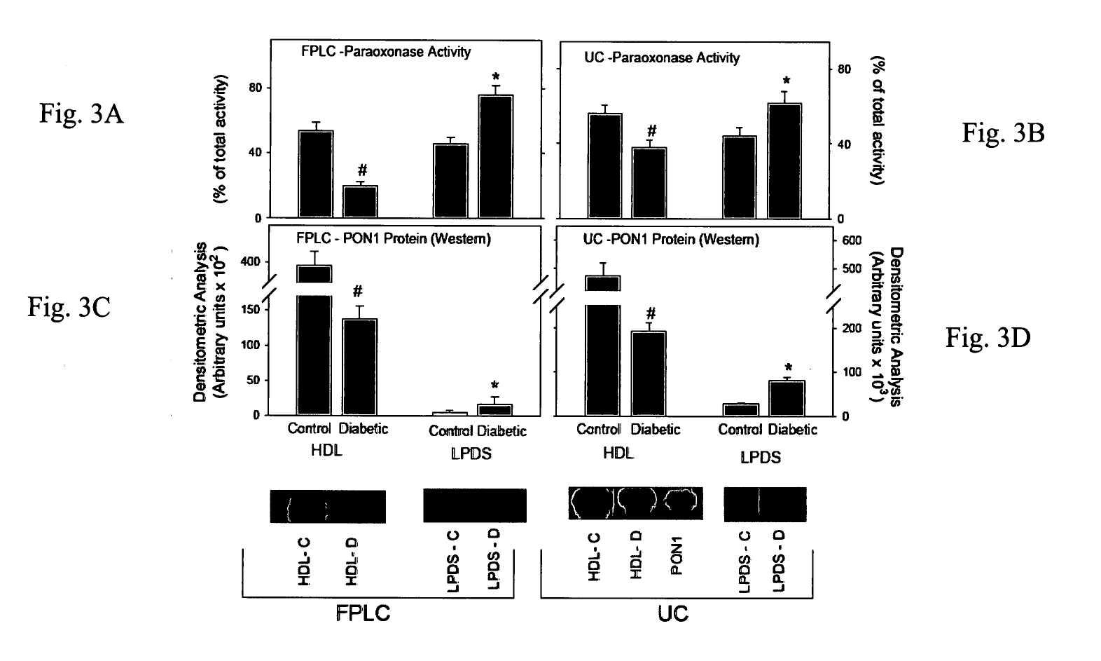

Whilst reducing the present invention to practice, the present inventors have shown that the distribution of both PON1 and apo-A1 between HDL and LPDS is altered in a diabetic state (FIGS. 3A-D). Paraoxonase activity in HDL from the diabetic patients was significantly reduced, compared to the control healthy HDL paraoxonase activity (FIG. 3A). In contrast, paraoxonase activity in the patients' LPDS fractions was significantly higher, as compared to the control healthy LPDS (FIGS. 3A-B).

The apoA-I levels in the diabetic patient's HDL samples were severely reduced compared with the apoA-I levels in controls HDL. In contrast, in the diabetic patient's LPDS apoA-I levels were significantly increased compared with apoA-I levels in control LPDS. As such, the present inventors concluded that PON1 distribution and/or apo-A1 distribution may serve as markers for diabetes and other related diseases.

Thus, according to one aspect of the present invention, there is provided a method of diagnosing a subject with a lipid related disorder, the method comprising determining an amount or activity of PON1 in an LPDS fraction of a serum of the subject, wherein an amount or activity of PON1 above a predetermined threshold is indicative of the lipid related disorder.

As used herein, the term “diagnosing” refers to classifying a disease or a symptom as a lipid-related disorder, determining a predisposition to a lipid related disorder, determining a severity of a lipid related disorder, monitoring disease progression, forecasting an outcome of a disease and/or prospects of recovery.

The phrase “lipid related disorder” as used herein, refers to a disorder which results in abnormal levels of lipids and/or cholesterol in the body. Many lipid related disorders are a result of abnormal levels or activities of HDL and are typically related to an increase in oxidative stress. Examples of lipid-related disorders include cardiovascular diseases including but not limited to atherosclerosis, hypercholesterolemia, coronary heart disease, myocardial infarction, peripheral vascular diseases, venous thromboembolism, stroke and pulmonary embolism. Other lipid related disorders include stroke, thrombotic disorder, transitory ischemic attacks, and lipoprotein abnormalities associated with Alzheimer's disease, obesity, diabetes mellitus, syndrome X and impotence.

The term “subject” as used herein, refers to a mammalian subject, preferably a human subject.

As used herein, the term “PON1” refers to paraoxonase-1 protein such as set forth in GenBank Accession No. NP_{—}000437 (e.g. version NP_{—}000437.3) and EC Nos. 3.1.1.2; 3.1.8.1

The present inventors have shown that an increase of PON1 in the LPDS fraction correlates with a decrease in PON1 in HDL. Without being bound to theory, the present inventors propose that fractionation of PON1 from the HDL towards LPDS results in oxidative stress and ultimately brings about symptoms of the disease. Thus, according to a preferred embodiment of the present invention, an amount or activity of PON1 is also measured in HDL. A ratio of PON1:HDL to PON:1 LPDS may be determined, wherein a ratio below a predetermined threshold is indicative of the lipid related disorder. Exemplary ratios are provided hereinbelow.

As used herein, the phrase “ratio of PON1:HDL to PON:1 LPDS” refers to a quantitative (i.e. amount of PON1)or qualitative (i.e. activity of PON1) ratio.

PON1 measurements of the present invention may be effected in vitro/ex-vivo in a serum sample. Various methods are known in the art for obtaining lipoprotein deficient serum and for isolating HDL. Two exemplary methods are described in the Example section below, discontinuous gradient ultracentrifugation and Fast-Protein Liquid Chromatography (FPLC).

Determining an amount of PON1 in the LPDS or HDL fraction is preferably effected at the protein level with the aid of PON1 specific antibodies.

The term “antibody” as used in the present invention includes intact molecules as well as functional fragments thereof, such as Fab, F(ab′)₂, and Fv that are capable of binding to macrophages. These functional antibody fragments are defined as follows: Fab, the fragment which contains a monovalent antigen-binding fragment of an antibody molecule, can be produced by digestion of whole antibody with the enzyme papain to yield an intact light chain and a portion of one heavy chain; Fab′, the fragment of an antibody molecule that can be obtained by treating whole antibody with pepsin, followed by reduction, to yield an intact light chain and a portion of the heavy chain; two Fab′ fragments are obtained per antibody molecule; (Fab′)₂, the fragment of the antibody that can be obtained by treating whole antibody with the enzyme pepsin without subsequent reduction; F(ab′)₂ is a dimer of two Fab′ fragments held together by two disulfide bonds; Fv, defined as a genetically engineered fragment containing the variable region of the light chain and the variable region of the heavy chain expressed as two chains; and single chain antibody (“SCA”), a genetically engineered molecule containing the variable region of the light chain and the variable region of the heavy chain, linked by a suitable polypeptide linker as a genetically fused single chain molecule.

Antibodies specific for PON1 are commercially available from such companies as Abcam, Cambridge, U.K. (e.g. catalogue number ab24261).

Methods of producing polyclonal and monoclonal antibodies as well as fragments thereof are well known in the art (See for example, Harlow and Lane, Antibodies: A Laboratory Manual, Cold Spring Harbor Laboratory, New York, 1988, incorporated herein by reference).

Exemplary methods for determining the amount of PON1 in LPDS or HDL are described hereinbelow.

Immunoprecipitation (IP) is the technique of precipitating an antigen (i.e. PON1) out of solution using an antibody specific to that antigen. The complexes are brought out of solution by insoluble antibody-binding proteins isolated initially from bacteria, such as Protein A and Protein G. These can also be coupled to sepharose beads that can easily be isolated out of solution. After washing, the amount of the precipitate can be analyzed using mass spectrometry, western blotting, or any number of other methods for identifying constituents in the complex.

Western blot: This method involves separation of a substrate (i.e. PON1) from other protein by means of an acrylamide gel followed by transfer of the substrate to a membrane (e.g., nylon or PVDF). Presence of PON1 is then detected by antibodies specific to PON1, which are in turn detected by antibody binding reagents. Antibody binding reagents may be, for example, protein A, or other antibodies. Antibody binding reagents may be radiolabeled or enzyme linked as described hereinabove. Detection may be by autoradiography, colorimetric reaction or chemiluminescence. This method allows both quantitation of an amount of substrate and determination of its identity by a relative position on the membrane which is indicative of a migration distance in the acrylamide gel during electrophoresis.

Radio-immunoassay (RIA): In one version, this method involves precipitation of the desired protein (i.e., PON1) with a specific antibody and radiolabeled antibody binding protein (e.g., protein A labeled with I¹²⁵) immobilized on a precipitable carrier such as agarose beads. The number of counts in the precipitated pellet is proportional to the amount of substrate.

In an alternate version of the RIA, a labeled substrate (i.e. PON1) and an unlabelled antibody binding protein are employed. A sample containing an unknown amount of substrate is added in varying amounts. The decrease in precipitated counts from the labeled substrate is proportional to the amount of substrate in the added sample.

As mentioned hereinabove, diagnosing lipid-related disorders may also be effected based on the activity (e.g. paraoxonase activity) of PON1 in LPDS alone or LPDS and HDL serum fractions.

As shown by the present inventors, soluble PON1 (i.e. PON1 present in the LPDS) loses its arylesterase and lactonase activities, but maintains its paraoxonase activity. The paraoxonase activity in LPDS, therefore, correlates with the amount of soluble PON1.

Measuring PON1 enzyme activity (e.g. paraoxonase activity) may be effected using a chromogenic substrate (e.g. paraoxon) which is not a substrated for PON1's other enzymatic activities. The paraoxonase activity of PON1 catalyzes a reaction in which the paraoxon is decomposed to produce a chromogenic product. The activity can be measured in a spectrophotometer well using colorimetric methods or can be measured in a non-denaturing acrylamide gel (i e., activity gel). Following electrophoresis the gel is soaked in a solution containing a substrate and colorimetric reagents. The resulting stained band corresponds to the enzymatic activity of the protein of interest. If well calibrated and within the linear range of response, the amount of enzyme present in the sample is proportional to the amount of color produced. An enzyme standard is generally employed to improve quantitative accuracy. A method of measuring paraoxonase activity is described in the Examples section hereinbelow.

In order to increase the reliability of the diagnosis method of the present invention, the amount of apo-A1 (Genbank accession no. NP_{—}000030) in HDL and LPDS may also be measured. Without being bound to theory, it is believed that the disassociation of PON1 from HDL is related to the disassociation of apo-A1 from HDL to LPDS and thus the migration of apo-A1 from HDL to LPDS during lipid related disorders such as diabetes mirrors the migration of PON1 from HDL to LPDS. Methods of measuring apo-A1 levels in LPDS and HDL are described hereinabove. Similar to PON1 antibodies, apo-A1 antibodies are also commercially available from vendors such as Abcam (e.g. cat. No.ab8945).

The agents which are used to measure PON1 or apoA quantity and/or activity may be presented in a kit. The kit of the present invention may, if desired, be presented in a pack which may contain one or more units of the kit of the present invention. The pack may be accompanied by instructions for using the kit and for fractionating serum so that LPDS and HDL fractions are generated. The pack may also be accommodated by a notice associated with the container in a form prescribed by a governmental agency regulating the manufacture, use or sale of laboratory supplements, which notice is reflective of approval by the agency of the form of the compositions.

As mentioned hereinabove, an amount or activity of PON1 above a predetermined threshold is indicative of the lipid related disorder. Furthermore, a PON1:HDL to PON1:LPDS ratio below a predetermined threshold is indicative of the lipid related disorder.

Determination of such predetermined thresholds may be effected on an empirical basis by analyzing a large number of serum samples from healthy subjects and patients already diagnosed with lipid-related disorders. Patient samples may also be categorized according to severity of disease such that thresholds may be determined for a particular stage of the disease.

Exemplary predetermined thresholds are provided hereinbelow.

LPDS/HDL Paraoxonase Activity Ratio

Controls (healthy): from about 1 to about 1.5 and more preferably from about 0.9 to about 1.4 and even more preferably from about 0.9 to about 1.3.

Patients: from about 1.5 to about 2.2 and more preferably from about 1.6 to about 2.2 and even more preferably from about 1.7 to about 2.2., −1.79±0.21

LPDS/HDL Amount (Densitometric Analyses) Ratio

Controls (healthy): from about 0.04 to about 0.08 and more preferably from about 0.04 to about 0.07 and even more preferably from about 0.04 to about 0.06.

Patients: from about 0.3 to about 0.5 and more preferably from about 0.4 to about 0.5 and even more preferably from about 0.45 to about 0.55

Serum LPDS Paraoxonase Activity

Controls (healthy): from about 30 to about 43 Units/ml and more preferably from about 30 to about 40 Units/ml and even more preferably from about 30 to about 38 Units/ml.

Patients: from about 45 to about 60 Units/ml and more preferably from about 48 to about 60 Units/ml and even more preferably from about 52 to about 60 Units/ml

Serum Apo A-I Amount (Densitometric Analyses)

Controls (healthy): from about 110 to about 240 mg/ml and more preferably from about 140 to about 240 mg/ml and even more preferably from about 170 to about 240 mg/ml.

Patients: from about 80 to about 125 mg/ml and more preferably from about 80 to about 110 mg/ml and even more preferably from about 80 to about 100 mg/ml

As used herein, the term “about” refers to ±10%.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions, illustrate the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N.Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorpotaed by reference as if fully set forth herein. Other general references are provided throughout this document. The procedures therein are believed to be well known in the art and are provided for the convenience of the reader. All the information contained therein is incorporated herein by reference.

General Materials and Methods

Subjects: Blood samples were collected from a control group consisting of 3 male healthy volunteers, 3 male NIDDM patients, and 3 male hypercholesterolemic patients. The controls were non smokers, with no diabetes (serum glucose levels below 100 mg % and hemoglobin Alc levels were in the range of 4.8-6.2%), with no hypertension, or coronary artery disease, without medications. The diabetes mellitus duration in the patients was 4-10 years with serum glucose levels above 160 mg % and hemoglobin Alc in the range of 7.5-11.3%. Both patient groups had no ischemic heart disease, no hypercholesterolemia and were non smokers.

The serum samples were immediately analyzed for glucose, lipids (cholesterol and triglyceride) apolipoprotein A-I levels, PON1 activities and basal oxidative status. Following analysis, the serum samples were fractionated.

Isolation of serum lipoproteins by Fast-Protein Liquid Chromatography (FPLC): Serum lipoproteins were separated by size exclusion chromatography on an FPLC system (22). Two hundred microliters of serum were diluted ×2 with phosphate buffered saline (PBS) and filtered. These diluted serum samples were fractionated through Superose 6 column (1×30 cm, Pharmacia) using prefiltered and degassed PBS pH 7.5. The flow rate was 0.5 ml/minute, and sixty 0.5 ml fractions were collected and immediately analyzed for PON1 arylesterase and paraoxonase activities. VLDL was eluted between fractions 15-17, LDL between 23-26, HDL between 27-36, and LPDS between 37-60. The HDL peak consisted of two peaks: the first one HDL₂ and the second one HDL₃.

Isolation of serum lipoproteins by discontinuous density gradient ultracentrifugation (UC): HDL and LPDS were prepared from human serum obtained from fasted normolipidemic volunteers, or diabetic patients by density gradient ultracentrifugation (23). Solid KBr was added to 4 ml serum to increase its density to 1.25 g/ml. This solution was overlayered with 4 ml of d=1.084 g/ml KBr—NaCl solution, and overlayered with 4 ml of d=1.006 g/ml. The KBr solutions contained 2 mM of CaCl₂ and 100 μM of diethylenetriaminepeta-acetic acid (DTPA) to preserve PON1 activity (24). The tubes were centrifuged in a SW41 rotor (Beckman Coulter Canada, Inc.) at 35,000 rpm (100,000 g) for 48 hours at 4° C. The VLDL, LDL, HDL and LPDS fractions were visualized, isolated and stored at 4° C. (23). The HDL and LPDS fractions were dialyzed against 50 mM Tris-HCL, 2 mM CaCl₂, pH 7.4, and their protein content was determined using the Folin phenol reagent (25).

Human PON1 preparation: PON1 (a generous gift from Dr. Draganov, University of Michigan, Ann Arbor, USA) was purified from the sera of healthy human volunteers, by chromatography using Blue Agarose, DEAE and Con-A columns, as previously described (26).

Evolved PON1 preparation: Evolved PON1 (a generous gift from Dr. Dan Tawfic from the department of Biological Chemistry, the Weitzman Institute of Science, Rehovot, Israel) was generated in E. coli after a directed evolution process as previously described (27).

PON1 Western Blot Analysis: Western blot analysis was performed using SDS-PAGE, 10% Bis-acrylamide gels. From the UC HDL or LPDS fractions, 20 μl were loaded on the gel, and from the FPLC fractions 150 μl were loaded. Blocking of the gel was in 2% BSA for 2 hours at room temperature. The primary antibody (a generous gift from Dr. Draganov, Ann Arbor, Michigan, USA) was mouse monoclonal anti-human PON1 diluted (1:5000, v/v in T-TBS with 1% BSA), and it was incubated with the membrane over night. The secondary antibody horseradish peroxidase-conjugated anti-mouse IgG (Sigma), diluted 1:5000 in T-TBS was incubated for 1 hour at room temperature. The membranes were developed using the ECL Western blotting kit (Amersham). Two micoliters (diluted 1:200) of purified hPON1 (3.8 mg/ml), were loaded as positive control. The membranes were exposed for 5 minutes.

Arylesterase activity: Arylesterase activity was determined using 5 μl serum or 10 μl of the HDL or LPDS fraction in a total volume of 1 ml of 50 mM Tris-HCL, pH 8.0 containing 1 mM CaCl₂ and 1 mM phenyl acetate. The increase in OD at 270 nm was monitored for 1 minute. One unit is defined as 1 μmol of phenyl actetate hydrolyzed per ml per minute (26).

Lactonase activity: Lactonase activity was determined using 10 μl of serum or 50 μl of HDL or LPDS fractions in a total volume of 1 ml of 50 mM Tris-HCL, pH 8.0 containing 1 mM CaCl₂ and 1 mM dihydrocoumarin. The increase in OD at 270 nm was than monitored along 1 minute. One unit is defined as 1 μmol of dihydrocoumarine hydrolyzed per ml per minute (3).

Paraoxonase activity: Paraoxonase activity towards paraoxon was determined spectrophotometrically at 412 nm using 10 μl serum or 50 μl of HDL or LPDS fractions (26). One unit of paraoxonase activity is defined as 1 nmol of 4-nitrophenol formed per minute.

Serum, HDL or LPDS lipid peroxidation: HDL or LPDS from healthy subjects (5 paraoxonase units/ml) were incubated with or without 100 mM of 2.2′-azobis, 2-amidinopropane hydrochloride (AAPH, Wako, Japan) for 2 hours at 37° C. (28). At the end of the incubation period, the amount of lipid peroxides (29) was measured. Basal serum lipid peroxidation status was measured by the thiobarbituric acid reactive substances (TBARS) assay (30).

Cells: J774 A.1 murine macrophage cells were purchased from the American Tissue Culture Collection (ATCC, Rockville, Md.). The cells were grown in Dulbecco's modified Eagle's medium (DMEM) containing 5% FCS and 100 U penicillin/ml, 100 μg streptomycin/ml, and 2 mM glutamine.

Macrophage Cholesterol efflux determination: J774 A.1 macrophages were incubated with [³H]-labeled cholesterol (2 μCi/ml) for 1 hour at 37° C., followed by cell wash in ice-cold PBS (×3) and a further incubation in the absence or presence of HDL or LPDS (5 paraoxonase units/ml)) for 3 hours at 37° C. (18). Cellular and medium [³H]-labeled cholesterol was quantitated and the percentage of cholesterol efflux was calculated [the ratio of [³H]-label in the medium*100/([³H]-label in the medium+[³H]-label in the cells), 18]. HDL or LPDS-mediated cholesterol efflux represents the value obtained in the presence of HDL or LPDS minus the value obtained in cells incubated without HDL or LPDS.

Statistics: Student's t test was performed for all statistical analyses. Results are given as mean±S.D.

### EXAMPLE 1

### Paraoxonasel (PON1) Distribution in Human Serum from Healthy Subjects and Diabetic Patients

Results

A representative fast protein liquid chromatography (FPLC) profile of serum lipoprotein fractions obtained from healthy subjects is shown (FIG. 1A). PON1 arylesterase and paraoxonase activities were measured in the VLDL, LDL, HDL and LPDS fractions (FIGS. 1B and 1C). Most of the serum arylesterase activity was recovered in the HDL fraction, with very low activity levels found in the VLDL and LDL fractions. Up to 12% of total serum arylesterase activity was found in the LPDS fraction (FIG. 1B). Similar results were obtained for serum lactonase activity (data not shown). In contrast to the data obtained for serum distribution of arylesterase and lactonase activities, a substantial paraoxonase activity was noted in the LPDS fraction which could account for 44% of total serum paraoxonase activity (FIG. 1C). Paraoxonase activity in the VLDL and LDL fractions was very low (FIG. 1C).

HDL and LPDS were also isolated from the healthy subjects by discontinuous density-gradient ultracentrifugation (UC). Similar to the FPLC fractions results, also in the UC LPDS fraction, paraoxonase activity was almost the same as that obtained in HDL (FIG. 2A). In contrast, arylesterase activity was lower in LPDS than in HDL by 78% (FIG. 2B), and lactonase activity was lower in LPDS than in HDL by 88% (FIG. 2C).

The above results from FPLC and UC clearly indicate that PON1 in LPDS loses its arylesterase and lactonase activities and maintain its paraoxonase activity, whereas in HDL, PON1 possesses all three activities.

In order to prove that the LPDS paraoxonase activity is indeed related to PON1, the PON1 specific inhibitor 2-hydroxyquinoline (200 μM) was used. The PON1 inhibitor resulted in a significant 60% reduction in HDL arylesterase activity (from 22.4±1.5 to 9.0±1.0 units/ml), and also in a significant 76% reduction in LPDS arylesterase activity (from 2.9±0.5 to 0.7±0.7 units/ml). Similarly, paraoxonase activities in HDL and LPDS were also significantly reduced by the addition of PON1 inhibitor or mouse anti human PON1 monoclonal antibody (diluted 1:10, v:v) to these fractions (data not shown). These results clearly indicate that the arylesterase/paraoxonase activity in LPDS, as well as in HDL is indeed related to PON1.

Under pathological conditions, the distribution of PON1 in serum between HDL and LPDS could have been altered, as was previously shown in patients with apoA-I deficiency [20, 21]. Thus, PON1 distribution was analyzed in serum from atherosclerotic patients, including diabetic patients. Increased oxidative stress was noted in the serum obtained from diabetic vs. healthy controls, since the basal serum TBARS levels in the diabetic patients were elevated by 116% compared with the controls (See Table 1, hereinbelow).

TABLE 1

| | Healthy | Diabetic |
|---|---|---|
| | Subjects | Patients |
| Oxidative Stress | 2.5 ± 0.2 | 5.4 ± 0.4* |
| nmol TBARS/ml | | |
| Glucose | 83 ± 6 | 350 ± 20* |
| mg % | | |
| Triglyceride | 113 ± 11 | 202 ± 5 |
| mg % | | |
| Total Cholesterol | 191 ± 12 | 155 ± 27 |
| mg % | | |
| LDL-Cholesterol mg % | 112 ± 21 | 88 ± 32 |
| HDL-Cholesterol | 50 ± 5 | 22 ± 3* |
| mg % | | |
| Apolipoprotein A-I | 170 ± 68 | 103 ± 10* |
| mg % | | |
| Paraoxonase Activity | 340 ± 37 | 183 ± 35* |
| U/ml | | |
| Arylesterase Activity | 113 ± 16 | 55 ± 7* |
| U/ml | | |
| Lactonase Activity | 22 ± 4 | 8 ± 3* |
| U/ml | | |

Serum samples were collected from 3 healthy subjects and 3 diabetic patients. Basal serum oxidative status was determined by the TBARS assay. Results represent meand ± SD (n = 3).
*p < 0.01 vs. Healthy Subjects.

Serum paraoxonase, arylesterase and lactonase activities were significantly reduced in the diabetic patients by 46%, 51%, and 64%, respectively, compared with the activities measured in the controls (Table 1). Serum apolipoprotein A-I (apoA-I) levels were significantly lower in the diabetic patients by 39% (Table 1). Serum HDL cholesterol levels were also significantly reduced in the diabetic patients by 56%, compared with the levels present in healthy subjects (Table 1).

Using FPLC or ultracentrifugation fractionation, the distribution of PON1 between HDL and LPDS in serum from diabetic patients was compared to that observed in healthy subjects (FIG. 3A-D). In the FPLC system, paraoxonase activity in HDL from the diabetic patients (expressed as % of total activity in HDL+LPDS), was significantly reduced, by 2.8 fold, compared to the control healthy HDL paraoxonase activity (FIG. 3A). In contrast, paraoxonase activity in the patients' LPDS fractions was significantly higher, by 1.7 fold, as compared to the control healthy LPDS (FIG. 3A). Similar results for paraoxonase activity were obtained upon serum fractionation by ultracentrifugation, with a 1.3 fold reduction in the diabetic patient HDL activity, and a 1.6 fold increase in the diabetic patient LPDS paraoxonase activity, compared with the activities observed in control healthy HDL or LPDS (FIG. 3B). ApoA-I concentrations in the controls group and the diabetic patients HDL and LPDS samples were measured. The apoA-I levels in the diabetic patient's HDL samples were reduced by 63%, compared with the apoA-I levels in controls HDL (3.5±0.3 vs. 9.5±2.4 mg/fraction). In contrast, in the diabetic patient's LPDS apoA-I levels were increased by 21% compared with apoA-I levels in control LPDS (0.33±0.12 vs. 0.27±0.06 mg/fraction). In the diabetic patients, 8.4% of total serum apoA-I concentration were found in the LPDS fraction, in comparison to only 3.2% that were noted in control's LPDS, indicating increased dissociation of apoA-I (like PON1) from HDL to the LPDS fraction in diabetes vs. controls. Paraoxonase activity in the diabetic patients HDL was reduced by 30%, in comparison to the activity observed in control's HDL when expressing it as units per mg of HDL apoA-I (data not shown).

In order to demonstrate that the paraoxonase activity in LPDS is indeed related to PON1, a Western blot analysis using PON1 monoclonal antibody was performed. In the FPLC system, the amount of PON1 protein in the diabetic patient HDL was significantly reduced by 2.8 fold as compared with the control healthy HDL (FIG. 3C). In contrast, the amount of PON1 protein in the diabetic patient LPDS was increased by 3.7 fold as compared to the healthy control LPDS (FIG. 3C). Similar results were obtained for the ultracentrifugation fractions; PON1 protein was reduced by 2.5 fold in the diabetic HDL, or increased by 2.9 fold in the diabetic LPDS, compared with the control HDL or LPDS, respectively (FIG. 3D). These results correlate with PON1 paraoxonase activity in the HDL or LPDS fractions obtained by both serum fractionation methods. In the UC fractions, the PON1 protein content was considerably lower in LPDS vs. HDL in both groups (controls and diabetic patients). However, the PON1 protein content in the control healthy LPDS was found to be about 5% of the total PON1 protein found in HDL+LPDS, whereas, in the diabetic patients, as much as 30% of the total PON1 protein in HDL+LPDS was found in their LPDS fractions (FIG. 3D).

The diabetic patient HDL (obtained by UC) contained 8.6±2.0 nmol lipid peroxides/ml compared with only 0.8±0.1 nmol/ml in the control healthy HDL (n=3), suggesting that the increased oxidative stress in the diabetic patient HDL may be the result of its reduced PON1 levels and activity.

The increase in PON1 in LPDS (both protein and activity) in the diabetic patients vs. healthy controls may be the result of a reduced capability of the diabetic HDL to bind PON1. To analyze this possibility, similar volumes of HDL from control or diabetic patient (separated by ultracentrifugation) were incubated with PON1 (50 paraoxonase units/ml) for 20 hours at 37° C., followed by a second ultracentrifugation in order to remove unbound PON1. Paraoxonase activity in the patient HDL (expressed as units per mg of apoA-I) was increased by only 4% as compared to 127% increment in control HDL (data not shown), suggesting that the patient HDL is indeed less capable of binding PON1.

### EXAMPLE 2

### PON1 in HDL but Not in LPDS Protects Against Lipid Peroxidation

Results

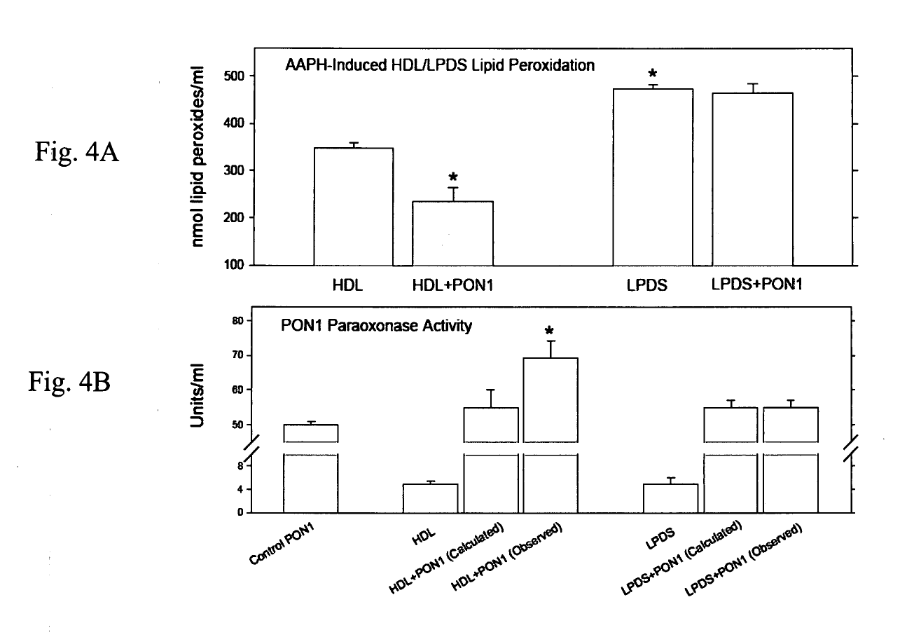

PON1 presence in HDL or in LPDS could have important consequences on PON1 antioxidant capability. Thus, the ability of PON1 in LPDS vs. HDL (obtained from controls) to protect against AAPH-induced lipid peroxidation was compared (FIG. 4A). HDL or LPDS (5 paraoxonase units/ml) was incubated both with and without 50 paraoxonase U/ml of evolved PON1 for 2 hours at 37° C. Upon addition of 100 mM AAPH, the extent of lipid peroxidation in LPDS, measured by the lipid peroxides assay, was increased by 36% compared with HDL. In HDL incubated with the PON1, compared with control HDL (incubated without PON1,) a 33% decrement in AAPH-induced lipid peroxides level was noted (FIG. 4A). In contrast, enrichment of LPDS with PON1 had no significant effect on LPDS lipid peroxidation (FIG. 4A). Similar results were observed with purified human PON1 (data not shown). These results indicate that PON1 in HDL, but not lipoprotein-free PON1 in LPDS protects against lipid peroxidation.

To analyze whether HDL, in comparison to LPDS can stimulate PON1 paraoxonase activity, HDL or LPDS (5 paraoxonase units/ml) were incubated with or without PON1 for 2 hours at 37° C., followed by paraoxonase activity measurement (FIG. 4B). The observed paraoxonase activity of PON1 in HDL was significantly higher by 26%, compared to the calculated values (paraoxonase activities in HDL+ that added as purified PON1, FIG. 4B). In contrast, the observed paraoxonase activity in LPDS was similar to the calculated value (FIG. 4B). These results suggest that HDL, but not LPDS, increases PON1 paraoxonase activity, probably by its ability to stabilize the enzyme.

### EXAMPLE 3

### PON1 in HDL Stimulates Cholesterol Efflux From Macrophages More than when Present in LPDS

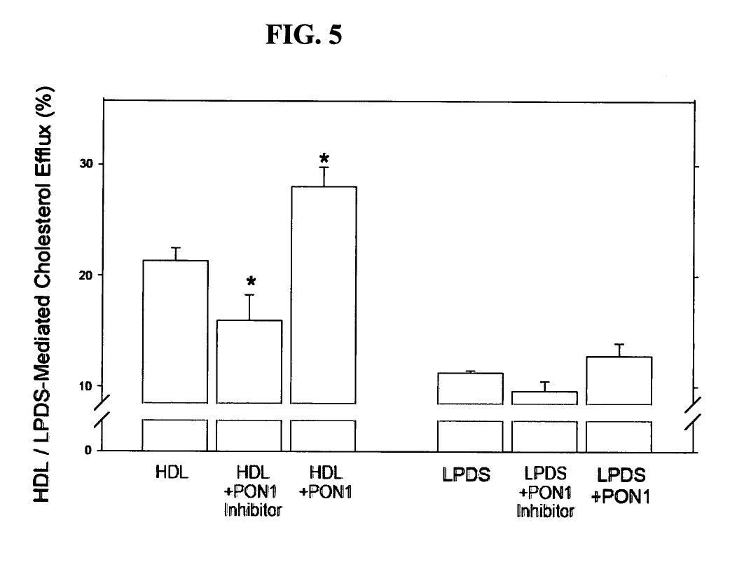

It has been shown by the present inventors that HDL-associated PON1 has a stimulatory role in HDL-mediated cholesterol efflux from macrophages [18]. Thus, the ability of PON1 in LPDS was compared to the ability of PON1 in HDL (obtained from 3 healthy subjects) to induce macrophage cholesterol efflux (FIG. 5). LPDS ability to induce cholesterol efflux from J774 A.1 macrophages compared to HDL (at a similar PON1 paraoxonase activity), was found to be 3.4 fold lower (FIG. 5). To analyze PON1 contribution to HDL/LPDS mediated macrophage cholesterol efflux, HDL or LPDS was preincubated with PON1 inhibitor 2-hydroxyquinoline (200 μM). Cholesterol efflux from the cells by HDL preincubated with the inhibitor was reduced by 25%, compared with the extent of cholesterol efflux by non-treated HDL (FIG. 5). In contrast, cholesterol efflux by LPDS preincubated with the PON1 inhibitor was reduced only by 10%, compared with the extent of cholesterol efflux by non-treated LPDS (FIG. 5). Furthermore, the HDL or the LPDS fractions were enriched with similar paraoxonase activity of purified PON1. Cholesterol efflux from J774 A.1 macrophages by HDL enriched with PON1 (50 U/ml of paraoxonase activity) was 31% higher than that observed with HDL (that was not enriched with PON1, FIG. 5). In contrast, LPDS enrichment with a similar purified PON1 paraoxonase activity, increased cholesterol efflux from the cells by only 13%, compared with the value obtained on using LPDS that was not enriched with PON1 (FIG. 5). These results suggest that PON1 in HDL can stimulate macrophage cholesterol efflux significantly more than PON1 in LPDS.

### CONCLUSIONS

The present study demonstrates, for the first time, that in diabetes a significant amount of serum PON1 is dissociated from HDL to the LPDS fraction. Furthermore, the present inventors have shown that PON1 in LPDS, unlike PON1 in HDL, is not able to protect against lipids peroxidation, and to stimulate macrophage cholesterol efflux.

Western blot analysis in serum fractions revealed [6, 7] PON1 presence mainly in HDL (>90%), but also in the triglyceride-rich lipoproteins chylomicrons (˜1%) and in VLDL (˜2%), and in the lipoprotein-deficient serum (LPDS, ˜5%) [21]. The present study confirmed a similar content of PON1 protein in LPDS separated by ultracentrifugation, but lower levels (˜1%) in LPDS prepared by FPLC. This phenomenon could be related to the ultracentrifugation forces that dissociate HDL surface constituents (apoA-I, phospholipids and also PON1) from the lipoprotein to LPDS.

An interesting finding of the present study is the significant paraoxonase activity observed in LPDS, up to similar levels as those observed in HDL. Previous study [22] showed by using FPLC that there was almost no arylesterase activity in the LPDS fraction, and concluded that PON1 is completely associated with HDL. That study however did not measure paraoxonase activity, which is considered to be a more specific activity of PON1 [31]. The present study also could not detect significant amount of arylesterase (as well as lactonase) activity in the LPDS fraction, but a substantial paraoxonase activity was measured in this fraction, suggesting that PON1 changes its conformation when present in LPDS which results in the loss of its arylesterase and lactonase activities, but stimulate its paraoxonase activity. It might be also that the lactonase/arylesterase activities of PON1 are more important than the paraoxonase activity to its physiological roles (in oxidation protection and cholesterol efflux), which are probably related to PON 1-association with HDL [32].

Western blot analysis, as well as the use of PON1 specific inhibitor, or the mouse anti human PON1 antibody, all provided clear evidence that PON1 protein is indeed present not only in HDL, but also in LPDS, and the observed paraoxonase activity in LPDS, as in HDL is related to PON1.

HDL-associated PON1 was previously shown to protect against lipid peroxidation [9,10,12-15], a phenomenon that was attributed to its ability to hydrolyze specific oxidized lipids in lipoproteins [13, 33], in macrophages [10,11] and in the atherosclerotic lesions [34]. The present inventors have recently demonstrated another antioatherogenic property of PON1. i.e.: its ability to stimulate macrophage cholesterol efflux [18].

The present study demonstrated that PON1 in LPDS vs. HDL loses its protection against LPDS or HDL lipid peroxidation, and its ability to stimulate macrophage cholesterol efflux. These phenomena could be related to the ability of HDL but not of LPDS, to stabilize PON1, secondary to the effect of apoA-I, the major apolipoprotein in HDL [1, 2], as well as HDL-associated specific phospholipids, which bind the PON1 N-terminal leader sequence [1].

The lipids in LPDS that can be oxidized are various free phospholipids, or in association with unesterified cholesterol, as well as albumin-bound free fatty acids. The LPDS cholesterol acceptor which is responsible for macrophage cholesterol efflux could be free apoA-I as well as phospholipids.

Diabetic patients are at high risk to develop accelerated atherosclerosis, which could be related to the increased oxidative stress observed in these patients [35-38].

In diabetic patients, serum paraoxonase, arylesterase and lactonase activities, were all significantly lower than the activities measured in control healthy subjects, Furthermore, apoA-I concentration in the diabetic patients was substantially reduced in comparison to controls. ApoA-I deficiencies in human and mice, were associated with a significant reduction in serum PON1 activities [1, 20-22]. Furthermore, as shown in the present study in diabetes, not only serum PON1 activities were reduced in comparison to control healthy subjects, but also the distribution of PON1 from HDL to LPDS was clearly demonstrated, as was shown previously in human and mice apoA-I deficiencies [20-22]. The dissociation of PON1 from the diabetic HDL to LPDS, may be the result of reduced apoA-I levels in HDL, of HDL lipid peroxidation, and also of HDL glycation, as glycation of HDL, as occurs in diabetes, was shown to inactivate PON1 [40]. The diabetic HDL was less able to bind PON1, even when expressing the activity per mg of HDL apoA-I, further indicating that the diabetic HDL (unlike normal HDL) has a poor capability of stabilizing PON1 activity.

The PON1 activity in the diabetic patients HDL was significantly reduced, compared with control's HDL even when expressing it per HDL apoA-I content, suggesting PON1 dissociation in diabetic patients from HDL to the LPDS. It is possible that in diabetic patients some of their apoA-I and PON1 are present on very high density lipoproteins which are heavier and much smaller particles in size than HDL₂ and HDL₃, or LPDS-associated PON1 could also be attached phospholipids.

It has been previously shown that PON1 protects against atherosclerosis, by its ability to reduce macrophage foam cell formation (via reducing oxidative stress and stimulation of cholesterol efflux from macrophages, 10, 12, 15, 18). The present study suggests that PON1 association with HDL is important for PON1 ability to perform its anti-atherogenic effects, and dissociation of PON1 from HDL to the LPDS fraction is accompanied by the loss of PON1 anti-atherogenic properties.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

### REFERENCES

### Other References are Cited in the Document

1. Sorenson R C, Bisgaier C L, Aviram M, et al. Human serum paraoxonase/arylesterase's retained hydrophobic N-terminal leader sequence associates with HDLs by binding phospholipids : apolipoprotein A-I stabilizes activity. Arterioscler Thromb Vasc Biol. 1999; 19: 2214-25.
2. James R W, Deakin S P. The importance of high-density lipoprotein for paraoxonase-1 secretion, stability, and activity. Free Radic Biol Med._{—}2004; 37:1986-94.
3. Teiber J F, Draganov D I, La Du B N. Lactonase and lactonizing activities of human serum paraoxonase 1 (PON1) and rabbit serum PON3. Biochem Pharmacol. 2003; 66:887-96.
4. Getz G S, Reardon C A. Paraoxonase, a cardioprotective enzyme: continuing issues. Curr Opin Lipidol. 2004; 15:261-67.
5. Mackness M, Durrington P N, Mackness B. Paraoxonase 1 activity, concentration and genotype in cardiovascular diseases. Curr Opin Lipidol. 2004; 15:399-404.
6. Fuhrrman B, Volkova N, Aviram M. Paraoxonase 1 (PON1) is present in postprandial chylomicrons. Atherosclerosis. 2005; 180:55-61.
7. Deakin S, Moren X, James R W. Very low density lipoproteins provide a vector for secretion of paraoxonase-1 from cells. Atherosclerosis. 2005; 179:17-25.
8. Harel M, Aharoni A, Gaidukov L, et va. Structure and evolution of the serum paraoxonase family of detoxifying and anti-atherosclerotic enzymes. Nat Struc Mol Biol. 2004; 11:412-19.
9. Shih D M, Xia Y R, Miller E, et al. Combined serum paraoxonase knockout/apolipoprotein E knockout mice exhibit increased lipoprotein oxidation and atherosclerosis. J Biol Chem. 2000; 275:17527-35.
10. Rozenberg O, Rosenblat M, Coelman R, et al. Paraoxonase (PON1) deficiency is associated with increased macrophage oxidative stress: studies in PON1-knockout mice. Free Radic Biol Med. 2003; 34:774-84.
11. Tward A, Xia Y R, Wang X P, et al. Decreased atherosclerotic lesion formation in human serum paraoxonase transgenic mice. Circulation. 2002; 106:484-90.
12. Rozenberg O, Shih S D, Aviram M. Paraoxonase 1 (PON1) attenuates macrophage oxidative status: studies in PON1 transfected cells and in PON1 transgenic mice. Atherosclerosis. 2005 (in press).
13. Aviram M, Rosenblat M, Bisgaier C L, et al. Paraoxonase inhibits high density lipoprotein (HDL) oxidation and preserves its functions: a possible peroxidative role for paraoxonase. J Clin Invest. 1998; 101:1581-90.
14. Mackness M I, Arrol S, Abbott C A, Durrington P N. Protection of low-density lipoprotein against oxidative modification by high-density lipoprotein associated paraoxonase. Atherosclerosis. 1993; 104:129-35.
15. Aviram M, Rosenblat M. Paraoxonases 1, 2, and 3, oxidative stress, and macrophage foam cell formation during atherosclerosis development. Free Radic Biol Med. 2004; 37:1304-16.
16. Fuhrman B, Volkova N, Aviram M. Oxidative stress increases the expression of the CD36 scavenger receptor and the cellular uptake of oxidized LDL in macrophages from atherosclerotic mice: protective role of antioxidants and of paraoxonase. Atherosclerosis. 2002; 161:307-16.
17. Rozenberg O, Shih D M, Aviram M. Human serum paraoxonase (PON1) decreases macrophage cholesterol biosynthesis: a possible role for its phospholipase-A₂ activity and lysophosphatidylcholine formation. Arterioscler Thromb Vasc Biol. 2003; 23: 461-67.
18. Rosenblat M, Vaya J, Shih D M, Aviram M. Paraoxonase 1 (PON1) enhances HDL-mediated macrophage cholesterol efflux via the ABCA1 transporter in association with increased HDL binding to the cells: a possible role for lysophosphatidylcholine. Atherosclerosis. 2005; 179:69-77.
19. Bergmeier C, Siekmeier R, Gross W. Distribution spectrum of paraoxonase activity in HDL fractions. Clin Chem 204: 50:1-7.
20. James R W, Blatter Garin MC, Calabresi L, et al. Modulated serum activities and concentrations of paraoxonase in high density lipoprotein deficiency states. Atherosclerosis. 1998; 139:77-82.
21. Noto H, Hashimoto Y, Satoh H, et al. Exclusive association of paraoxonase 1 with high-density lipoprotein particles in apolipoprotein A-I deficiency. Biochem Biophys Res Commun. 2001; 289:395-401.
22. Cabana V G, Reardon C A, Feng N, et al. Serum paraoxonase: effect of the apolipoprotein composition of HDL and the acute phase response. J Lipid Res. 2003; 44:780-92.
23. Aviram M. Plasma lipoprotein separation by discontinuous density gradient ultracentrifugation in hyperlipoproteinemic patients. Biochem Med. 1983; 30:111-18.
24. Connelly P W, Maguire G F, Draganov D I. Separation and quantitative recovery of mouse serum arylesterase and carboxyesterase activity. J Lipid Res. 2004; 45:561-66.
25. Lowry O H, Rosenberg N J, Farr L, Randall R J. Protein measurement with the Folin phenol reagent. J Biol Chem. 1951; 193:265-25.
26. Gan K, Smolen A, Eckerson H W, La Du B N. Purification of human serum paraoxonase/arylesterase: evidence for one esterase catalyzing both activities. Drug Metab Dispos. 1991; 19:100-106.
27. Aharoni A, Gaidukov L, Toker L, et al. Directed evolution of mammalian paraoxonases PON1 and PON3 for bacterial expression and catalytic specialization. Proc Natl Acad Sci. 2003; 101:482-87.
28. Frei B, Stocker R, Ames B N. Antioxidant defenses and lipid peroxidation in human blood plasma. Proc Natl Acad Sci USA. 1988; 85:9748-52.
29. El-Saadani M, Esterbauer N, El-sayed M, et al. Spectophotometric assay for lipid peroxides in serum lipoproteins using commercially available reagent. J Lipid Res. 1989; 30:627-30.
30. Buege J A, Aust A D. Microsomal lipid peroxidation. Methods Enzymol. 1978; 52:302-10.
31. Draganov D I, Teiber J F, Speelman A, et al. Enzymatic characterization or recombinant human paraoxonases (PON1, PON2 and PON3)—lactonases with overlapping and distinct substrate specificities. J Lipid Res. 2005; in press.
32. Aviram M, Rosenblat M. Paraoxonases (PONs) and cardiovascular diseases: pharmacological and nutritional influences. Curr Opin Lipidol. 2005, in press.
33. Ahmed Z, Ravandi A, Maguire GF et al. apolipoprotein A-I promotes the formation of phosphatidylcholine core aldehydes that are hydrolyzed by paraoxonase (PON1) during high density lipoprotein oxidation with peroxynitrite donor. J Biol Chem. 2001; 276:24473-81.
34. Aviram M, Hardak E, Vaya J, et al. Human serum paraoxonase (PON1), Q and R selectively decrease lipid peroxides in coronary and carotid atherosclerotic lesions: PON1 esterase and peroxidase-like activities. Circulation. 2000; 101:2510-17.
35. Letellier C, Duron M R, Jouanolle A M, et al. Serum paraoxonase activity and paraoxonase gene polymorphism in type 2 diabetic patients with or without vascular complications. Diabetes Metab. 2002; 28:297-304.
36. Tsuzura S, Ikeda Y, Suchiro T, et al. Correlation of plasma oxidized low-density lipoprotein levels to vascular complications and human serum paraoxonase in patients with type 2 diabetes. Metabolism. 2004; 53:297-302.
37. Ferretti G, Bacchetti T, Busni D, et al. Protective effect of paraoxonase activity in high-density lipoproteins against erythrocyte membranes peroxidation: a comparison between healthy subjects and type 1 diabetic patients. J Clin Endocrinol Metab. 2004; 89:2957-62.
38. Rosenblat M, Hayek T, Hussein K, Aviram M. Decreased macrophage paraoxonase 2 expression in patients with hypercholesterolemia is the result of their increased cellular cholesterol content: effect of atorvastatin therapy. Arterioscler Thromb Vasc Biol. 2004; 24:175-80.
39. Aviram M, Rosenblat M, Billecke S, et al. Human serum paraoxonase (PON1) is inactivated by oxidized low density lipoprotein and preserved by antioxidants. Free Radic Biol Med. 1999; 26: 892-904.
40. Ferretti G, Bacchetti T, Marchionni C, et al. Effect of glycation of high density lipoproteins on their physiochemical properties and on paraoxonase activity. Acta Diabetol. 2001; 38:163-69.

What is claimed is:

1. A method of diagnosing a subject with a lipid related disorder, the method comprising determining an amount or activity of PON1 in an LPDS fraction of a serum of the subject, wherein an amount or activity of PON1 above a predetermined threshold is indicative of the lipid related disorder.

2. The method of claim 1, further comprising determining an amount or activity of PON1 in an HDL fraction of said serum.

3. The method of claim 1, wherein said activity of PON1 is a paraoxonase activity.

4. The method of claim 3, further comprising determining an amount of apo-A1 in said LPDS fraction of said serum of the subject.

5. The method of claim 4, further comprising determining an amount of apo-A1 in an HDL fraction of said serum.

6. The method of claim 1, wherein the lipid related disorder is selected from the group consisting of diabetes mellitus, atherosclerosis, coronary heart disease, myocardial infarction, peripheral vascular diseases, venous thromboembolism and pulmonary embolism.

7. The method of claim 6, wherein the lipid related disorder is diabetes mellitus.

8. A method of diagnosing a subject with a lipid related disorder, the method comprising determining an amount of apo-A1 in an LPDS fraction of a serum of the subject, wherein an amount of apo-A1 above a predetermined threshold is indicative of the lipid related disorder.

9. The method of claim 8, further comprising determining an amount of apo-A1 in an HDL fraction of said serum.

10. The method of claim 8, further comprising determining an amount or activity of PON1 in said LPDS fraction of said serum of the subject.

11. The method of claim 10, further comprising determining an amount or activity of PON1 in an HDL fraction of said serum.

12. The method of claim 8, wherein the lipid related disorder is selected from the group consisting of diabetes mellitus, atherosclerosis, coronary heart disease, myocardial infarction, peripheral vascular diseases, venous thromboembolism and pulmonary embolism.

13. The method of claim 12, wherein the lipid related disorder is diabetes mellitus.

14. A method of diagnosing a subject with a lipid related disorder, the method comprising determining a ratio of an amount or activity of PON1:HDL to PON1:LPDS in a serum of the subject, wherein a ratio below a predetermined threshold is indicative of the lipid related disorder.

15. The method of claim 14, wherein said activity of PON1 is a paraoxonase activity.

16. The method of claim 14, wherein the lipid related disorder is selected from the group consisting of diabetes mellitus, atherosclerosis, coronary heart disease, myocardial infarction, peripheral vascular diseases, venous thromboembolism and pulmonary embolism.

17. The method of claim 16, wherein the lipid related disorder is diabetes mellitus.

18. The method of claim 15, further comprising determining a ratio of an amount of apo-A1:HDL to apo-A1:LPDS in said serum of said subject.

19. A kit for diagnosing or determining a predisposition to a lipid related disorder, the kit comprising at least one agent capable of determining an amount or activity of PON1 in a LPDS fraction of a serum sample and instructions for carrying out the diagnosing in said LPDS fraction.

20. The kit of claim 19, wherein the lipid related disorder is selected from the group consisting of diabetes mellitus, atherosclerosis, coronary heart disease, myocardial infarction, peripheral vascular diseases, venous thromboembolism and pulmonary embolism.

21. The kit of claim 20, wherein the lipid related disorder is diabetes mellitus.
